(19) United States
(12) Patent Application Publication
(10) Pub. No.: US 2026/0240434 A1
(43) Pub. Date: Aug. 20, 2026

(54) SYSTEM FOR MONITORING AT LEAST ONE PARAMETER REPRESENTATIVE OF A DECOMPENSATION OF A CARDIAC FAILURE BY MEANS OF A SUBCUTANEOUS IMPLANT

(71) Applicant: CARDIAMETRICS, Caen (FR)

(72) Inventors: Cindy MICHEL, La Tronche (FR); Quentin ROZAND, La Tronche (FR)

(73) Assignee: CARDIAMETRICS, Caen (FR)

(21) Appl. No.: 19/146,276

(22) PCT Filed: Jan. 31, 2024
(86) PCT No.: PCT/FR2024/050128

§ 371 Date: Jul. 8, 2025

(30) Foreign Application Priority Data
Jan. 31, 2023 (FR) FR2300910

(51) Int. Cl. A61B 5/00; A61B 5/01; A61B 5/0205; A61B 5/07; A61B 5/08

(52) U.S. Cl. CPC A61B 5/0006; A61B 5/0022; A61B 5/0031; A61B 5/01; A61B 5/02055; A61B 5/07; A61B 5/0816; A61B 2562/0219; A61B 2562/0271

(57) ABSTRACT

A system for monitoring at least one parameter representative of an episode of decompensation, the monitoring system comprising at least one subcutaneous implant, the subcutaneous implant comprising at least one electrocardiograph and one accelerometer configured to collect information relating at least to the functioning of the heart of the living being, the monitoring system also comprising a computer server and a communication relay configured to allow at least the exchange of information collected by the subcutaneous implant with the computer server, the computer server being configured to calculate changes in at least one parameter, from among hemodynamic and/or respiratory and/or electrophysiological parameters, on the basis of the data collected by at least the accelerometer and the electrocardiograph.

The present invention relates to the field of medical devices and systems for monitoring the cardiac health of a living being. The present invention relates more particularly to a system for monitoring parameters characteristic of a decompensation of a cardiac failure, wherein an implantable medical device communicating with a computer server is configured to measure cardiac parameters.

The cardiac failure is a chronic condition affecting a large proportion of the population, particularly the over-60s, and its frequency increases with the age of the patient. As the population ages, the incidence of cardiac failure is set to rise by around 25% every four years.

The episodes of decompensation of the cardiac failure are often accompanied by emergency hospitalization of the patient suffering from this cardiac condition. Indeed, the episodes of decompensation of the cardiac failure often appear as asymptomatic at their onset and are not detected early enough. The episode of decompensation of the cardiac failure progresses and the patient begins to feel the first symptoms of the decompensation of the cardiac failure: a fatigue, palpitations and a shortness of breath. When these symptoms appear, there is a high probability that the patient will need to be admitted to hospital as an emergency to stabilize their condition.

The decompensations of a cardiac failure may be detected before the appearance of symptoms by in-depth analysis of various sub-clinical cardiac parameters, particularly hemodynamic parameters. Detecting a decompensation of a cardiac failure in a patient in this way allows the patient to be treated by prescribing a therapeutic treatment that stabilizes the cardiac function of the patient without the need for hospitalization.

The present invention is part of this context and is intended to provide a system for monitoring at least one parameter representative of an episode of decompensation of a cardiac failure, which is particularly capable of providing information relating to changes in a cardiac function of a patient from one monitoring period to the next. This information will allow a healthcare professional to assess the state of health of the patient and, on the basis of this information, decide whether or not it is necessary to intervene. Where appropriate, the monitoring system may be equipped with an algorithm configured to help the healthcare professional make decisions on the basis of said information.

Thus, the main purpose of the present invention is a system for monitoring at least one parameter representative of an episode of decompensation of a cardiac failure in a living being, the monitoring system comprising at least one subcutaneous implant configured to be introduced under the skin of said living being, the subcutaneous implant comprising at least one electrocardiograph and one accelerometer configured to collect data relating at least to the functioning of the heart of the living being, the monitoring system also comprising a computer server and a communication relay configured to allow at least the exchange of data collected by the subcutaneous implant with the computer server, the computer server being configured to calculate changes in at least one parameter, from among hemodynamic and/or respiratory and/or electrophysiological parameters, on the basis of the data collected by the accelerometer and the electrocardiograph, the subcutaneous implant comprising communication means configured to communicate data collected by at least the accelerometer and the electrocardiograph by means of a low-energy Bluetooth connection. The subcutaneous implant is a medical device that is implanted under the skin of a patient suffering from chronic cardiac failure, and more specifically implanted close to the heart. In particular, this subcutaneous implant comprises an accelerometer and an electrocardiograph allowing for collecting data relating to changes in the state of health of the patient, and in particular for defining an information representative of changes in these parameters by combining the data collected over several days, it being understood that changes in at least one parameter in unusual proportions may be indicative of an episode of decompensation. It is understood that the monitoring system is able to monitor the state of health of the patient via changes in at least one parameter from among hemodynamic parameters, respiratory parameters and electrophysiological parameters, so as to provide early evidence of the occurrence of an episode of decompensation of a cardiac failure.

By means of the data transmitted by the subcutaneous implant, the monitoring system, in particular via the computer server, is able to treat information allowing to monitor changes in various cardiac markers, both hemodynamic and electrophysiological, in particular the pre-ejection period, the amplitude of cardiac sounds, the width of the QRS complex, and/or changes in respiratory markers such as respiratory frequency. The monitoring system is thus able to transmit to appropriate medical personnel a plurality of items of information relating, in particular, to changes in cardiac function. This information allows a practitioner to monitor the change of a cardiac failure of the patient and may allow to initiate a treatment as soon as a cardiac decompensation appears, even before the patient presents the symptoms of this cardiac decompensation.

The communication means allow the subcutaneous implant to communicate data relating to the cardiac function and/or to the respiratory function to an equipment located outside the body of the patient, for example the communication relay and/or the computer server, while limiting energy consumption by the subcutaneous implant. The issue of energy autonomy for the subcutaneous implants is essential in this field, as it ensures that patients avoid having to undergo surgery to replace the implant for as long as possible.

A low-energy Bluetooth connection, known by the acronym BLE for “Bluetooth Low Energy”, is used in a variety of applications, particularly for transmitting encryption keys between two elements that need to be connected. In other words, this low-energy connection has the advantage of being energy-efficient, but may only transmit small amounts of data, of the order of a few hundred bytes.

In the application of the invention, the BLE connection is also used to communicate data collected by the subcutaneous implant, which has a larger dimension. In particular, these data are data relating to a cardiac signal, collected by an accelerometer or an electrocardiogram, and they are of the order of several thousand bytes.

According to a characteristic of the invention, the communication means are configured to also communicate alerts relating to the availability of data collected by at least the accelerometer and the electrocardiogram communications separate from the data communication. It is clear that the problem of the size of the information transmitted via the

BLE connection does not arise for alerts, which consist of a message of just a few bytes. According to one characteristic of the invention, a communication protocol for the communication of data between the subcutaneous implant and the communication relay and/or the computer server is configured so that communication of data from the implant is carried out in a plurality of successive sessions for sending partial data. The communication between the communication means of the subcutaneous implant with the communication relay and/or with the computer server by means of a low-energy Bluetooth connection allows to limit the power consumption of the subcutaneous implant and therefore optimize the service life of the subcutaneous implant. This type of communication solution is particularly advantageous when implemented with a particular communication protocol, as a communication using a low-energy Bluetooth connection only allows a small volume of data to be exchanged. A communication protocol of this kind also allows the subcutaneous implant to send alerts towards the communication relay and to transmit all the data collected by the subcutaneous implant, even though the volume of data is large.

More particularly, each data item collected by the accelerometer and/or the electrocardiogram is split into a plurality of partial data items, the juxtaposition or the combination of which forms said collected data item, and the communication protocol implemented between the subcutaneous implant and the communication relay and/or the computer server consists of sending each of these partial data items in succession. It is understood that this partial data is of the order of a byte or a hundred bytes, a size that is conducive to the low-energy communication implemented by the invention for the data communication from the subcutaneous implant.

The data collected by the subcutaneous implant may thus be reconstructed by juxtaposing the partial data received successively by the communication relay and/or the computer server. According to a characteristic of the invention, the data collected by the subcutaneous implant is reconstructed by combining the partial data received successively by the communication relay and/or the computer server, the combination sequence being defined between the subcutaneous implant and the communication relay and/or the computer server. In other words, particularly for reasons of security when transferring medical data, it is possible to configure the subcutaneous implant so that the data communication is encrypted, with the data only being able to be correctly reconstructed if the sequence for combining the partial data is known to the communication relay and/or the computer server. This combination sequence may always be the same between this implant and the relay and/or the computer server, and thus not be revealed during the communication, or it may be changed at each data communication, one of the partial data items sent containing an information about the combination sequence.

As mentioned, the communication protocol allows the subcutaneous implant to communicate the data collected by means of a plurality of successive sessions for sending partial data, using a low-energy Bluetooth connection. Advantageously, the partial data is sent one after the other. According to a characteristic of the invention, the communication protocol is configured so that a partial data item includes at least one item of information relating to the number of partial data items forming said data communication. According to a characteristic of the invention, said partial data including the information relating to the number of partial data forming said data communication includes only this information. According to a characteristic of the invention, the partial data sent successively comprises said partial data including information relating to the number of partial data forming said data communication as well as one or more other partial data comprising at least some of the data collected by the subcutaneous implant.

For example, if a data collected by the subcutaneous implant is of the order of a thousand bytes and the data needs to be split into “n” partial data in order to transmit the entire data via a BLE connection, the communication protocol is configured so that “n+1” partial data are communicated, with a first partial data comprising the information that “n” partial data will follow, and the “n” subsequent partial data each comprising a part of the collected data. According to a characteristic of the invention, a communication protocol for the communication of data between the subcutaneous implant and the communication relay is different from a communication protocol for the exchange of data between the communication relay and the computer server.

When transferring data originally collected by the accelerometer or the electrocardiograph of the subcutaneous implant, the communication protocol, and more particularly the number of interactions, implemented for the communication between the subcutaneous implant and the communication relay is different from the communication protocol, and more particularly the number of interactions, implemented for the communication between the communication relay and the computer server.

The Bluetooth low-energy communication is implemented to limit the energy consumption of the subcutaneous implant and increase its service life. This consideration is less important for the communication relay, which may comprise a more powerful external battery or be connected directly to an electrical network for its power supply. The communication relay may also communicate the information transmitted by the subcutaneous implant, for example, by means of a WIFI connection allowing a larger volume of data to be exchanged than a low-energy Bluetooth connection and over a greater distance.

In other words, according to one characteristic of the invention, the BLE connection is specific to the communication between the subcutaneous implant and the communication relay, the communication between the communication relay and the computer server taking place via a high-speed communication network that allows data to be communicated over a considerable distance and also allows large data to be transferred.

For example, all the data collected by the subcutaneous implant for a given acquisition period is transferred from the subcutaneous implant to the communication relay in around ten successive coded messages, while the same data is then sent towards the computer server from the communication relay in a single message.

According to one characteristic of the invention, the subcutaneous implant comprises a temperature sensor configured to measure the body temperature of the living being. It is understood that the temperature sensor is used to measure the body temperature of the patient in whom the subcutaneous implant is implanted. In particular, this temperature sensor may be used to detect an immune reaction, a probable sign of an infection and a precipitating factor in the decompensation of a cardiac failure. This type of immune reaction is particularly evident when the body temperature is above 38° C.

According to one characteristic of the invention, the subcutaneous implant comprises at least one case on which at least a first electrode and a second electrode are arranged. These first and second electrodes are used to transmit data on the electrical functioning of the heart of the patient in whom the subcutaneous implant is implanted. The first and second electrodes are electrically conductive surfaces for collecting an electrical signal. This electrical signal is transmitted to an electrocardiogram a printed circuit board. The electrocardiograph calculates the potential difference between the first and second electrodes to obtain electrophysiological data such as cardiac rate and its variability, the width of the QRS complex and the duration of the QT segment. In addition, the data acquired by the electrocardiograph by calculating said potential difference may be coupled with data acquired by the accelerometer to obtain complementary data such as the pre-ejection period, i.e. the period between the electrical depolarization of a ventricle and the start of ventricular ejection. It should be noted that this pre-ejection period represents the period of contraction of the left ventricle for a constant volume of blood in the left ventricle at each contraction.

In addition, the first electrode and the second electrode may also be used to measure bioelectrical impedance values. These electrodes are able to emit and receive a low-intensity electric current and an associated control module, here on board the printed circuit board housed in the case, configured to measure the resistance of the biological tissues through which the current passes between an emitting electrode and a receiving electrode. More specifically, this characteristic of the electrodes to emit and receive a low-intensity electric current allows to obtain data on the changes in blood volumes that occur during the cardiac cycle. It is understood that this data may be used to obtain data on systolic time intervals.

In addition, the measurement of the bioelectrical impedance values by means of said electrodes also allows to obtain data on respiratory parameters such as tidal volume. This tidal volume represents the lung capacity at rest of the patient during a normal inspiration, i.e. when the patient is not making any particular effort, and gives an indication of the volume of air entering the lungs during an inspiration. A decrease in this tidal volume may indicate the presence of fluid in the lungs, a likely sign of a decompensation episode of the cardiac failure.

According to a characteristic of the invention, the computer server is configured to calculate at least one change relating to cardiac sounds and/or a pre-ejection period and/or a respiratory rate and/or a cardiac rate by means of the data collected by the subcutaneous implant.

According to one characteristic of the invention, the accelerometer is capable of calculating linear accelerations along three orthogonal axes, the computer server being configured to calculate changes in at least one hemodynamic and/or respiratory parameter on the basis in particular of a data of the accelerometer relating to at least one of said axes.

According to an alternative characteristic of the invention, the accelerometer is a simple accelerometer, configured to detect a linear acceleration along a single axis, the computer server being configured to calculate the changes in at least one hemodynamic and/or respiratory parameter on the basis in particular of a data acquired on one axis by the accelerometer.

According to one characteristic of the invention, an algorithm is implemented on the computer server, said algorithm being configured to analyze the data acquired and transmitted by the subcutaneous implant so as to assess the risk of occurrence of an episode of decompensation of the cardiac failure.

The invention also relates to a communication method for a monitoring system as previously mentioned, the communication method implementing:
at least a first step during which the communication relay scans the surrounding space to detect the subcutaneous implant,
at least a first additional step during which the subcutaneous implant generates a warning signal specifying that data relating to the operation of the heart is capable of being exchanged with the computer server,
at least one second step during which data or control instructions are exchanged between the subcutaneous implant and the computer server by means of the communication relay, the low-energy wireless communication network implemented for the communication between the subcutaneous implant and the communication relay being distinct from the high-speed wireless communication network implemented for the communication between the communication relay and the computer server.

The communication relay is able to scan the surrounding space over a radius of approximately 5 meters around the communication relay in search of a signal emitted by the subcutaneous implant. This detection distance from the communication relay limits the possibility of foreign devices connecting to the communication relay.

According to a characteristic of the invention, the communication method implements at least one second auxiliary step occurring before the second step and during which the communication relay detects the subcutaneous implant and sends to the computer server a request for identifying the subcutaneous implant, the computer server treating said identification request by sending to the subcutaneous implant, via the communication relay, an encryption key specific to said subcutaneous implant and a data retrieval request and/or a control instruction.

It is understood that the computer server “treats” the identification request insofar as the computer server is configured to use the identification request to identify the subcutaneous implant and choose the action to be carried out as a function of this identification of the subcutaneous implant. When the computer server has recognized the subcutaneous implant via the identification request, the computer server may decide that no action is necessary, for example that data feedback from the implant is not required, so that the communication is not established between the implant and the computer server. An information to this effect may be sent to the implant via the communication relay. When the computer server has recognized the subcutaneous implant via the identification request, the computer server may decide that a feedback data acquired by the implant is necessary to send or that the sending of new operating parameters is necessary to send to the implant. The computer server is then configured to transmit, in addition to the data retrieval instruction and/or the control instruction, a unique encryption key specific to the implant so that the implant may recognize that the communication is indeed coming from an authorized computer server and is not an attempt to pirate data.

The control instruction sent to the subcutaneous implant may contain instructions to modify the operating parameters of the subcutaneous implant, for example the data acquisition frequency.

It should be noted that the identification request sent by the subcutaneous implant comprises information that is specific to that subcutaneous implant. For example, the identification request may comprise the serial number of the subcutaneous implant and the MAC address of the BLE connection implemented by the subcutaneous implant. This identification request allows the computer server to recognize the subcutaneous implant that has been detected by the communication relay, and to adapt the sending of information to the subcutaneous implant, such as control instructions and/or a data retrieval request, as a function of the subcutaneous implant that has been recognized.

In one embodiment of the communication method, the data collected by the subcutaneous implant passes through the communication relay, i.e. the data is not stored in the communication relay. In other words, it is understood that, in this version of the communication method, the communication relay allows the data collected by the subcutaneous implant to be transmitted between said subcutaneous implant and the computer server and that it does not treat said data collected by the subcutaneous implant. This offers an advantage in terms of data security, as the communication relay does not have to be protected as effectively as, for example, the computer server.

According to a characteristic of the invention, the communication relay is configured to be able to temporarily store data coming from the subcutaneous implant. In other words, in a standard operating mode, i.e. with no malfunction of the communication network between the communication relay and the computer server and no malfunction of the BLE connection between the communication relay and the subcutaneous implant, the communication relay does not store the data, whereas in a degraded operating mode, with a potential or proven malfunction of the means of communication, the communication relay temporarily stores the data until the means of communication are re-established.

It is understood that in this version of the communication method, the communication relay is able to retain the data collected by the subcutaneous implant in the event that the computer server is unavailable and therefore in the event that the transfer of information from the communication relay towards the computer server is impossible. This version of the communication method ensures that the data collected by the subcutaneous implant is not lost if the computer server is unavailable.

According to another characteristic of the invention, the communication relay is able to temporarily store at least information transmitted by the computer server comprising a control instruction and an encryption key specific to a subcutaneous implant, allowing the communication relay to connect to this subcutaneous implant.

“Temporarily” means that the communication relay retains the information transmitted by the computer server until the next connection between the communication relay and the subcutaneous implant.

In this embodiment, it is understood that when the communication relay detects the presence of the subcutaneous implant and said subcutaneous implant signals that it has data to transmit, the communication relay is able to establish the connection with the subcutaneous implant without the intermediary of the computer server.

This version of the communication method has the advantage of being able to retrieve data collected by the subcutaneous implant or transmit the control instructions to the subcutaneous implant, for example, when the computer server is unavailable, i.e. when the communication relay is unable to communicate with the computer server.

In this version of the communication method, the communication relay is able to store an encryption key specific to the subcutaneous implant with which the relay is to communicate, and it is understood that this communication relay comprises appropriate means for decrypting this encryption key and communicating it to the subcutaneous implant. In this context, at least the means of decrypting the encryption key are protected by encryption means. Such a version has the advantage of being able to retrieve the data collected by the subcutaneous implant independently of the operation of the computer server.

It should also be noted that regardless of the version of the communication method implemented, the data collected by the subcutaneous implant and transmitted to the computer server and the information transmitted by the computer server to the subcutaneous implant are encrypted.

According to one characteristic of the invention, the communication between the communication relay and the subcutaneous implant is provided by a low-energy Bluetooth connection and the communication between the communication relay and the computer server is provided by a high-speed Internet connection. The Bluetooth connection minimizes the amount of energy stored in the subcutaneous implant, but on the other hand requires more data to be sent between the subcutaneous implant and the communication relay so that the latter may retrieve all the data acquired by the subcutaneous implant. Depending on the role assigned to the communication relay, it may transmit the data received from the subcutaneous implant directly to the computer server, or the communication relay may concatenate the data received successively from the subcutaneous implant over a given period and send the concatenated data to the computer server in a single transmission.

In accordance with a characteristic of the invention, the communication relay is configured to connect to a single subcutaneous implant, the communication relay being configured to continuously scan the surrounding space for seeking said subcutaneous implant.

According to one characteristic of the invention, the communication relay is able to detect a plurality of subcutaneous implants, each subcutaneous implant being configured to allow or prevent the connection of the communication relay to said subcutaneous implant as a function of the recognition by said subcutaneous implant of an encryption key, said encryption key being specific and unique to each subcutaneous implant, said encryption key being communicated by the computer server to said subcutaneous implants via the communication relay.

It is understood that the encryption key is a means of ensuring that the device wishing to connect to the subcutaneous implant is authorized by the monitoring system.

According to a characteristic of the invention, the subcutaneous implant may be configured to accept to establish a connection only with those communication relays for which it has their identifier in memory, for example the serial number and/or the mac address of the BLE connection. In another embodiment, the subcutaneous implant may be configured to accept attempts from any communication relay except those on a blacklist comprising the relays that have made a first attempt to connect to the subcutaneous implant without authorization from the computer server, i.e. without the appropriate encryption key.

Other characteristics, details and advantages of the invention will become clearer on reading the following description, on the one hand, and examples of embodiments given by way of indication and without limitation with reference to the attached schematic drawings, on the other hand, wherein:

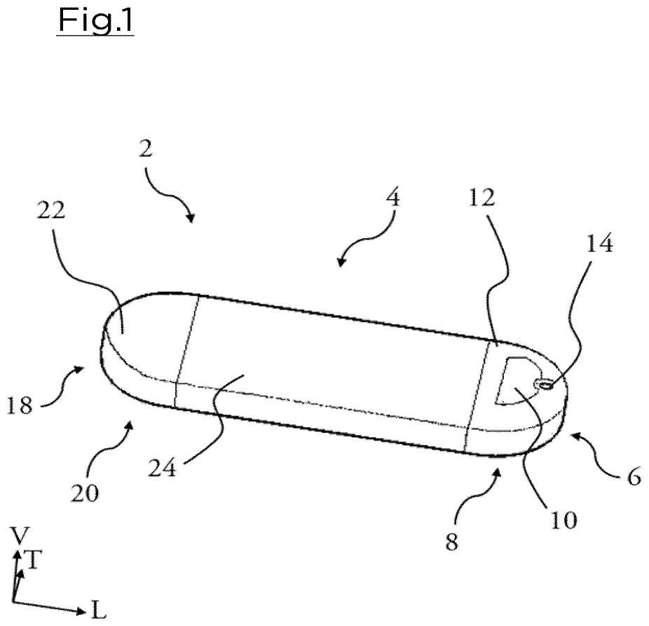

FIG. 1 shows a general view of a subcutaneous implant according to one embodiment of the invention;

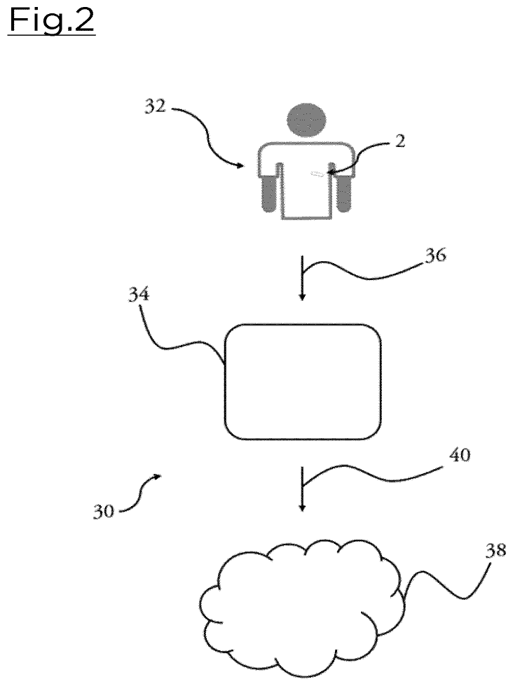

FIG. 2 represents very schematically the operation of a system for monitoring at least one parameter representative of an episode of decompensation of a cardiac failure in a patient, said patient being equipped with a subcutaneous implant forming part of the monitoring system in accordance with the invention;

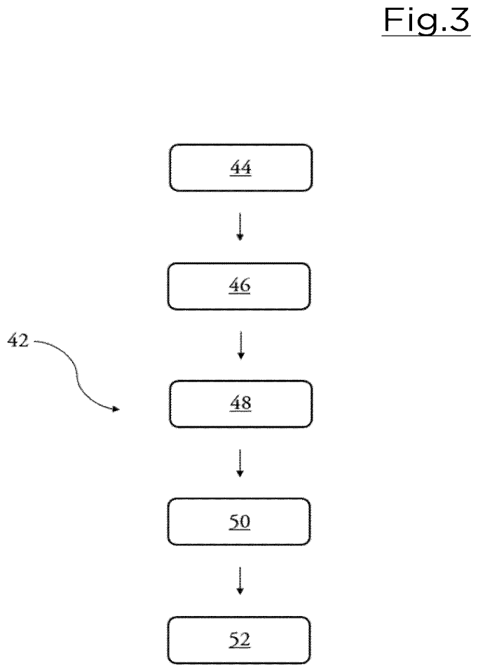

FIG. 3 is a schematic representation of an embodiment of a communication method implemented in the monitoring system.

First of all, it should be noted that although the figures set out the invention in detail for its implementation, these figures may of course be used to better define the invention, if necessary. It should also be noted that these figures only show examples of embodiments of the invention.

The characteristics, the variants and the different embodiments of the invention may be associated with one another in various combinations, insofar as they are not incompatible or mutually exclusive. In particular, it will be possible to imagine variants of the invention comprising only a selection of characteristics described hereinafter in isolation from the other characteristics described, if this selection of characteristics is sufficient to confer a technical advantage or to differentiate the invention from the prior art.

In the figures, the elements common to several figures retain the same reference.

FIG. 1 illustrates a medical device that may be implanted in a living being, and more specifically a subcutaneous implant 2 configured to be inserted under the skin of a living being, hereinafter referred to as the “patient”. In the embodiment shown, this subcutaneous implant 2 is located under the skin of the patient in the thoracic region between the 4^{th} intercostal space and the 5^{th} intercostal space, at the level of the left edge of the sternum.

Such a positioning of the subcutaneous implant 1 allows optimum measurements to be obtained both for the cardiac electrical parameters and for the cardiac mechanical parameters. It should be noted that in an alternative embodiment of the invention, the subcutaneous implant 1 may be located in particular on the 5^{th} intercostal space, at the level of the anterior axillary line or on the 4^{th} intercostal space at the level of the midclavicular line.

As may be seen in FIG. 1, the subcutaneous implant 2 extends in a main longitudinal direction of elongation, parallel to the axis L. More specifically, FIG. 1 shows a first face 4 of the subcutaneous implant 2.

The subcutaneous implant 2 comprises a first measuring end 8 at the level of a first longitudinal end 6. This first measuring end 8 comprises a first electrode 10, here arranged on the first face 4. By way of an illustrative and non-limiting example of the invention, this first measuring end 8 is formed in a polymer product 12 such as an epoxy polymer, and the first electrode 10 is arranged on the surface within this polymer, which forms an electrically insulating environment around the first electrode 10. It is understood that this first electrode 10 is electrically connected to a printed circuit board present in the subcutaneous implant in order to be able to retrieve an electrical potential information and/or to be able to emit a low-intensity current, but that the polymer product provides the electrical insulation with respect to the rest of the metal parts of the implant, in particular with respect to a second electrode, which will be described in more detail in the following description. Insulating the first electrode 10 and the second electrode in this way allows an electronic chip on board the subcutaneous implant 2 to measure a potential difference between these two electrodes and form an electrocardiograph.

The first measurement end may be made of another material, provided that this material allows the first electrode to be electrically insulated in accordance with the above and the subcutaneous implant 2 to be positioned under the skin of the patient without risk to the patient. Alternatively, the first measuring end may be made of a metallic material like the rest of the subcutaneous implant, for example titanium, provided that an insulating coating, for example of parylene, and an electrically insulating material extending through the thickness of the case are provided around the first measuring end exposing the first electrode.

In addition, this first measuring end 8 comprises an antenna, not shown here, capable of transmitting the information collected by the subcutaneous implant 2, as will be described in more detail in the following description.

The subcutaneous implant 2 comprises an orifice 14 arranged at the level of the first end 6 of the subcutaneous implant 2. Advantageously, this orifice 14 allows the subcutaneous implant 2 to be attached under the skin of the patient by means of a link. More specifically, this orifice 14 allows a link, such as a thread, to be passed through the orifice which, in the embodiment shown, passes through the subcutaneous implant 2 from the first face 4 to a second face, opposite the first face and not visible here. The thread is then attached to the biological tissue of the patient to maintain the subcutaneous implant 2 as it has been implanted.

The subcutaneous implant 2 comprises a second measuring end 18 at the level of a second end 20 opposite the first end 6. This second measuring end 18 comprises a second electrode 22 which, in combination with the first electrode 10 and an electronic chip as mentioned above, forms an electrocardiograph.

It should be noted that for an optimal operation of the subcutaneous implant 2, it is advantageous to implant the subcutaneous implant 2 in such a way that the first electrode 10 and the second electrode 22, and more generally the first face 4, face the heart of the patient.

Between the first end 6 and the second end 20, the subcutaneous implant 2 comprises a case 24 comprising at least one printed circuit board, an accelerometer and the electronic chip to which the first electrode 10 and the second electrode 22 are electrically connected to form the electrocardiograph. Advantageously, the case 24 also comprises a temperature sensor to highlight, for example, a potential infection in the patient increasing the risk of decompensation of the cardiac failure.

The case 24 comprises a first part housing an electrical energy storage means sized to ensure an appropriate autonomy for the subcutaneous implant and a second part housing, in particular, the accelerometer and the chip of the electrocardiograph mentioned above. In the example shown, each of these parts of the case and the two ends on which the electrodes are arranged are formed by hulls made independently of each other and welded together in a second stage. Alternatively, some of the parts between the case parts and the measuring ends may be made in one piece. By way of example, in an alternative embodiment of the invention, not shown here, the case may be formed in one piece from the first end to the second end and configured to house the battery and the printed circuit board.

In each of these cases, the first electrode 10 and the second electrode 22 are advantageously arranged at opposite ends of the subcutaneous implant.

Without limiting the invention, the case may be made of titanium and electrical insulation means are implemented to electrically insulate the electrodes 10, 22 integrated into the case 24 on either side of the case from each other.

In other words, the electrical insulation means ensure that there are no conductive elements connecting the first electrode 10 and the second electrode 22.

It should be noted that, without leaving the context of the invention, the first electrode and the second electrode may be insulated from each other by a different insulating material, preferably a polymer, as long as the latter does not present any health risk for the patient in whom the subcutaneous implant 2 is implanted.

In addition, the subcutaneous implant 2 comprises, as previously mentioned, the electrical energy storage means housed in the case 24. This electrical energy storage means may be a primary battery, such as an electric cell, or a rechargeable accumulator battery. The electrical energy storage means is configured to supply electrical energy to the various elements of the subcutaneous implant 2, particularly the printed circuit board.

As previously mentioned, at least one accelerometer, an electronic chip of an electrocardiograph and a temperature sensor are housed in the case 24, with the electrocardiograph electrodes arranged on the case 24.

In one embodiment, the accelerometer may be a three-axis accelerometer, capable of detecting linear accelerations along a longitudinal axis, a transverse axis and a sagittal axis. An average acceleration value may be calculated by normalizing the three values respectively detected on one of the axes. In an alternative embodiment of the invention, the accelerometer may be a simple accelerometer configured to detect a linear acceleration along a single axis.

The data obtained by the accelerometer is used in particular to analyze the hemodynamic and respiratory parameters of the patient, as well as an information relating to the posture of the patient at the time the information is taken by the subcutaneous implant 2, i.e., for example, whether the patient is standing or lying down, with an estimate of the angle of inclination of the lying position of the patient. In fact, during an episode of decompensation of a cardiac failure, the patient has difficulty breathing when he is lying down with zero inclination, i.e. flat. To overcome this difficulty, the torso of the patient is gradually straightened using additional pillows, with the change in inclination being detected by the accelerometer. It is understood that this information relating to the inclination of the patient, particularly during its sleep, allows a practitioner to determine, by cross-referencing this information with various markers if necessary, whether or not it is necessary to intervene.

As previously mentioned, the subcutaneous implant 2 is equipped with an electrocardiograph comprising the electronic chip located on the printed circuit board and first and second electrodes 10 and 22 arranged on the case 24. The combination of the electrocardiograph with the three-axis accelerometer advantageously facilitates the analysis of the data from the latter, particularly for the analysis of the cardiac sounds or of the pre-ejection period commonly known as “PEP”.

The temperature sensor measures the temperature of the patient. This temperature sensor provides information about the temperature of the patient in a more stable way than an external temperature measurement. This information about the temperature of the patient may be used to identify any immune reaction in the patient, indicating a potential infection. During an infectious episode, the patient is more exposed to the risk of cardiac decompensation. We understand that this data relative to the temperature of the patient allows the practitioner in charge of monitoring the patient to be more vigilant about the signals from the three-axis accelerometer and the electrocardiograph.

The operation of these on-board equipment in the subcutaneous implant, i.e. the electrocardiograph, the accelerometer and the temperature sensor, is advantageously controlled by the printed circuit board, and more specifically by a microcontroller. It is understood that the microcontroller is able to activate the on-board equipment in the subcutaneous implant in order, for example, to acquire data at a pre-set frequency. It should be noted that when the subcutaneous implant 2 is assembled, the printed circuit board is embedded in resin or silicone, by way of non-limiting examples. It is understood that this resin or silicone provides a rigid connection between the accelerometer and the case, so that all the movements perceived by the accelerometer are representative of the movements of the body involving the movement of the subcutaneous implant and may therefore be used for the treatment of the signal by the monitoring system.

The frequency at which measurements are taken is defined by a control system on a remote computer server. This frequency may be modified over time to adapt to the measures required to monitor the condition of the patient.

More specifically, the frequency of measurement of the subcutaneous implant 2 is, in a nominal mode of use, one data acquisition per day. However, the practitioner in charge of the patient may modify the number of daily acquisitions to monitor more precisely any disturbances or changes in the condition of the patient over the course of the day. Alternatively, the practitioner may choose to reduce the number of daily data acquisitions carried out by the subcutaneous implant 2 when, for example, the state of health of the patient is stable.

It is understood that the number of daily data acquisitions carried out by the subcutaneous implant 2 may be modulated by the practitioner as required. It should be noted that in the case of a single acquisition of data per day, this acquisition is advantageously carried out at night while the patient is asleep. In this way, the data collected by the subcutaneous implant 2 is not disrupted, for example, by physical activity such as climbing stairs. More specifically, the data acquisition by the subcutaneous implant 2 is advantageously carried out at a fixed time during the night when the patient is in a stable state that may be repeated day after day.

In addition, the subcutaneous implant 2 acquires a data over a time interval of approximately 30 seconds. Such a long data acquisition period allows the subcutaneous implant 2 to acquire data over a sufficiently large number of cardiac or respiratory cycles to ensure that it obtains information allowing a practitioner to analyze the risk of decompensation of the cardiac failure accurately. The duration of the data acquisition performed by the subcutaneous implant 2 may be longer, for example of the order of 2 minutes, to improve the accuracy of the information collected. It should be noted that the duration of the data acquisition may be modulated by the practitioner according to the precision of the information required. In addition, the duration of the data acquisition performed by the subcutaneous implant 2 may be longer than 2 minutes, depending on the amount of data that may be stored in the subcutaneous implant 2.

In addition, the printed circuit board comprises communication means allowing the subcutaneous implant 2 to transmit and/or receive information by means of the aforementioned antenna. More specifically, these communication means are wireless communication means using the Bluetooth® telecommunications standard, and more precisely the Bluetooth Low Energy protocol, better known by the acronym “BLE”.

As mentioned above, this subcutaneous implant 2 is configured to collect data autonomously, i.e. without the intervention of the patient, and this data is treated and stored in a computer server communicating with the subcutaneous implant to provide a practitioner with an information about changes in a particular parameter over the acquisition periods. By analyzing the changes in at least one parameter representative of a cardiac decompensation, the practitioner may assess the risk of a decompensation of the cardiac failure in the patient implanted with the subcutaneous implant 2. It should be noted that in the embodiment shown, in particular the embodiment shown in FIG. 2 which will be described later, the subcutaneous implant 2 is implanted in a patient suffering from chronic cardiac failure. Of course, in an alternative embodiment of the invention, the subcutaneous implant 2 may be implanted in a healthy patient not suffering from cardiac failure, the subcutaneous implant 2 may allow to detect the appearance of a cardiac failure.

The computer server is configured to provide the practitioner, such as a doctor in charge of the patient, with changes in the parameters determined from the data collected and transmitted by the subcutaneous implant 2. In particular, the practitioner may analyze changes in the cardiac sounds, changes in the duration of the pre-ejection period (PEP), changes in the respiratory frequency and/or, without limiting the invention, changes in the inclination of the patient during sleep.

The computer server may be configured to communicate all the information in its possession, i.e. all the changes in parameters that it has been able to calculate on the basis of all the data acquired by the subcutaneous implant. The accumulation of cardiac and/or respiratory markers whose change is assimilated to a deterioration, allows the practitioner to highlight the risk of an episode of cardiac decompensation.

Alternatively, the computer server may be configured, in particular when the state of health of the patient has been declared stable, to communicate to the practitioner only information about changes in a particular parameter, and in particular changes in markers that are known to be the first to deteriorate in the event of cardiac decompensation, such as changes in the amplitude of the cardiac sounds and changes in the duration of the pre-ejection period. In other words, the computer server may be configured to select the information sent to the practitioner, and to only send information on the evolution of other parameters when it has been judged that the evolution of early markers is indicative of the possible appearance of an episode of cardiac decompensation likely to occur.

For example, the amplitude of the cardiac sounds may be analyzed on the basis of the treatment of the accelerometric signal. This treating of the signal is advantageously carried out remotely, on the computer server, once the latter has retrieved all the data collected by the subcutaneous implant over a given acquisition period. The treating of the signal may consist of splitting the accelerometric signal into time cycles, each time cycle being analyzed to identify characteristic segments of the cardiac sound, known as segments S1, S2, S3. The amplitude of the segments identified in each time cycle is averaged so that three cardiac sound values are calculated for each acquisition period. By comparing these average cardiac sound values from one acquisition period to another, the computer server is configured to define a change in the amplitude of the cardiac sounds. The practitioner may thus receive, on an appropriate display means, an information relating to an increasing or decreasing trend in a particular cardiac sound S1, S2, S3. For example, an increase over time in the sound S1 and/or a decrease over time in the sound S3 may be an indicator of cardiac decompensation.

In addition to those mentioned above, one of the markers whose change is analyzed, may be the respiratory rate. During the episode of decompensation of a cardiac failure, the lung may fill with fluid. The tidal volume used by the patient with each breath is then lower, so the patient naturally compensates for this reduction in the volume of air used during normal breathing by increasing his or her respiratory frequency.

Another of these markers may be the inclination of the patient during its sleep. In fact, it is known that during an episode of decompensation, the patient presents respiratory difficulties that he compensates for by slightly raising his upper body during sleep, so the analysis of the evolution of the inclination of the patient during sleep is rich in information.

The cross-analysis of these different parameters allows the practitioner to define with a great precision the risk of decompensation of a cardiac failure in the patient and to intervene before the patient has to be hospitalized.

FIG. 2 shows a system 30 for monitoring an episode of decompensation of a cardiac failure. Within this monitoring system 30, the subcutaneous implant 2 is configured to collect measurements relating to the functioning of the heart of a patient 32 in whom the subcutaneous implant 2 is implanted.

In addition, the subcutaneous implant 1 is configured to communicate with a communication relay 34 so that the information containing the various measurements collected by the subcutaneous implant 2 may be transmitted from the subcutaneous implant 2 to the communication relay 34. This communication relay 34 is an electronic case located outside the body of the patient or a mobile application on a mobile apparatus such as a smartphone or tablet. The subcutaneous implant 2 communicates by means of a low-energy Bluetooth® connection 36 with the communication relay 34, sending messages with a limited amount of data, of the order of 241 bytes. The communication between the communication relay 34 and the subcutaneous implant 2 may thus be achieved by a plurality of messages, each comprising some of the information collected by the subcutaneous implant 2. Security measures are implemented to ensure the consistency and the confidentiality of all the information collected by the subcutaneous implant 2.

More specifically, the subcutaneous implant 2 is able to generate a signal or an alert to allow the implant to be detected by the communication relay 34. It should be noted that this step of detecting the subcutaneous implant 2 will be described in more detail in the following description. Once the subcutaneous implant 2 has been detected by the communication relay 34 and connected to the latter, the subcutaneous implant 2 is able to implement a specific communication protocol by means of communication means to allow it to communicate, via a low-energy connection, all the data collected by the items of equipment on-board in the subcutaneous implant 2.

This communication protocol is specific to the subcutaneous implant 2 and to the communication means of the subcutaneous implant 2. In particular, implementing a low-energy BLE connection to communicate medical data involves breaking down the data collected by the accelerometer and/or the electrocardiogram into “n” partial data, which takes the form of a signal over a given time cycle and is therefore one or several thousand bytes in size.

In particular, the communication protocol may be configured so that, when a data has to be communicated to the communication relay, the subcutaneous implant 2 sends a first message, or a first partial data, comprising a specific information relating to the number of messages, or partial data, that the subcutaneous implant 2 will send in order to transmit all the data collected. This first message initiates a transmission sequence comprising as many messages as necessary for the subcutaneous implant 2 to transmit all the data collected. It should be noted that communicating all the data collected by the subcutaneous implant 2 in a plurality of messages, or partial data, requires a prior step of fragmenting the information collected by the subcutaneous implant 2 into a plurality of these partial data. It should also be noted that the communication relay and/or the computer server is also configured to be able to reconstruct the data from the partial data, either by juxtaposing them in the order of reception, or by combining them according to a combination sequence specific to this data communication.

The data transfer using the Bluetooth low power protocol requires a relative proximity between the subcutaneous implant 2 located inside the body of the patient 32 and the communication relay 34 located outside the body of the patient 32. To this end, the communication relay 34 may advantageously be located, for example, in the bedroom of the patient 32 wearing the subcutaneous implant 2 so that data exchange may take place during the night when the patient 32 is located close to the communication relay 34.

The communication relay 34 is also configured to communicate with a computer server 38. At the level of this computer server 38 the information collected by the subcutaneous implant 2 are treated. More specifically, within the computer server 38, the information collected by the subcutaneous implant 2 is analyzed so as to define, for example, an amplitude of cardiac sounds.

According to the invention, the subcutaneous implant 2 is capable of transmitting data relating to the electrical functioning of the heart of the patient 32 to the computer server 38, by means of the low-energy Bluetooth connection 36 and the communication relay 34, this data being acquired by means of the electrocardiograph, the accelerometer and, where applicable, the temperature sensor. This information may be read, either directly or after a treatment of the appropriate signal, by a practitioner who may thus have, for carrying out a possible diagnosis of cardiac decompensation, information relating to the cardiac sounds S1, S2 and S3 in connection with systole and diastole, the cardiac rate, the cardiac rate variability, the width of the complex QRS, the duration of the segment QT, the cardiac rate and the pre-ejection period. It is understood that some of this data is advantageously deduced by cross-referencing information from the three-axis accelerometer and the electrocardiograph.

It should be noted that the information relating to the evolution of a parameter representative of a cardiac decompensation, as calculated and stored on the computer server 38, is communicated to the practitioner by information display means which may consist of the computer screen of the practitioner, the computer being connected to the server remotely via an Internet connection, or a mobile communication apparatus, such as a smartphone or tablet. It is understood that the computer server 38 is advantageously delocalized, i.e. hosted remotely, so that it may be accessed by a multitude of practitioners as long as they have secure access to retrieve the data relating to their patients and only their patients. Alternatively, without modifying the operation described above, the computer server 38 may be considered to be the computer or mobile communication apparatus of the practitioner, this computing or telecommunications equipment then comprising a software capable of performing the actions described above in relation to the computer server.

The information transmitted on the display means of the computing support or by means of mobile telecommunication may be transmitted in its entirety without prior treating or with a selection of the information to be provided to the practitioner. This selection can, for example, limit the transmission of the sensitive data depending on the type of display means used.

It should be noted that, additionally, the computer server 38 may comprise an algorithm capable of carrying out a first analysis of the data collected by the subcutaneous implant 2 and generating an alert for the practitioner if the information resulting from the treating of this data indicates a change deemed characteristic of a risk of cardiac decompensation.

The control system operating on the computer server 38 recognizes and validates the subcutaneous implant 2.

The computer server 38 may communicate, by means of the communication relay 34, with the subcutaneous implant 2 so as to modify the acquisition frequency and/or the time slot for these acquisitions and/or the type of data to be collected. By way of example, the computer server may generate a specific data retrieval request in the direction of the subcutaneous implant 2 or only process to a temperature acquisition, either immediately after receiving the specific data retrieval request, or during the next programmed acquisition session.

In the embodiment shown, the communication between the communication relay 34 and the computer server 38 is ensured via a secure internet connection 40. More specifically, this internet connection 40 is a WIFI connection. It is understood that the exchange of information between the communication relay 34 and the computer server 38 is dependent on the Internet connection 40.

It should be noted that in an alternative embodiment of the invention, it is possible for the computer server 38 to transmit data to the communication relay 34. This communication relay 34 may temporarily store the data transmitted by the computer server 38 until it establishes a communication with the subcutaneous implant 2 and transmits said information to it. This information may, for example, comprise changes to the frequency of data acquisition by the subcutaneous implant 2. Alternatively, the communication relay 34 may store the information transmitted by the subcutaneous implant 2, for example, in the event that the computer server 38 is not available. This information stored by the communication relay 34 will then be transmitted to the computer server 38 when it becomes available again.

The communication relay 34 is able to detect all the subcutaneous implants 2 in a surrounding space of around 5 meters. However, for the communication relay 34 to be able to connect to one of the subcutaneous implants 2, the communication relay 34 must provide this implant with an encryption key transmitted by the computer server 38. This encryption key is unique to each subcutaneous implant 2. It should be noted that the communication relay is not necessarily aware of the key, depending on the mode of communication implemented, but is able to present it to the subcutaneous implant.

In this context of communication to be established between a subcutaneous implant and a communication relay, the subcutaneous implant 2 may be configured to store in memory the communication relay or relays 34 to which it may connect and/or to blacklist all devices that have attempted to connect without authorization from the computer server 38.

In a particular secure mode of operation, the subcutaneous implant may be configured to connect to a single communication relay 34. This subcutaneous implant 2 is then uniquely associated with said communication relay 34.

If a communication relay 34 is configured so as to be able to connect to several subcutaneous implants 2 and act as a relay between each of these implants and the computer server, for example when several people in the same household are equipped with a subcutaneous implant of a monitoring system as described above, it is understood that the communication relay communicates with a single subcutaneous implant at a time, using the encryption key specific to each subcutaneous implant as supplied by the computer server 38 at the time of the acquisition period defined for this subcutaneous implant.

In particular, the monitoring system 30 allows the implementation of a communication method 42 specific to the invention during which, in a succession of steps, the computer server 38 is allowed to receive the desired information from the subcutaneous implant 2.

This communication method 42 may be seen more clearly in FIG. 3. As may be seen in FIG. 3, the communication method 42 implements a first step 44 during which the communication relay 34 scans the surrounding space for the subcutaneous implant 2. More specifically, it is understood that the communication relay 34 is fixedly positioned outside the body of the patient 32 and is continuously searching for a signal emitted by the subcutaneous implant 2.

In a first additional step 46, the subcutaneous implant 2 generates a warning signal allowing the communication relay 34 to detect the subcutaneous implant 2 and specifying that physiological data may be exchanged with the computer server 38. It should be noted that the subcutaneous implant 2 generates a signal at regular intervals, to allow the subcutaneous implant 2 to be detected by the communication relay 34 when the subcutaneous implant 2 is in proximity to the communication relay 34. The length of the transmission interval of the signal may be set, for example 5 minutes or 20 minutes. Like the signal emitted during the first additional step 46, this signal may comprise an indicator allowing the communication relay 34 to be aware that data is available and may be exchanged by the subcutaneous implant 2. This signal may also not comprise an indicator, in which case, however, the communication relay 34 may connect to the subcutaneous implant 2 if, for example, the computer server 38 has transmitted information to the communication relay 34 configured for the subcutaneous implant 2.

The activation of the Bluetooth and the emission of signals from said subcutaneous implant 2 requires energy. To increase the longevity of the energy storage means of the subcutaneous implant 2, the latter is connected to the communication relay 34 only, on the one hand when the subcutaneous implant 2 comprises information to be transmitted to the computer server 38 by means of the communication relay 34, and on the other hand when the communication relay 34 holds information emanating from the computer server 38 and configured for the subcutaneous implant 2.

The communication method 42 implements a second auxiliary step 48 which occurs, in the embodiment shown, when the communication relay 34 has detected the subcutaneous implant 2 and the latter has emitted an indicator indicating that one or more measurement information are suitable for retrieval by the computer server 38. During this second auxiliary step 48, the communication relay 34 detects the subcutaneous implant 2 and the computer server 38 generates a communication protocol comprising an information allowing the communication relay 34 to connect to the subcutaneous implant 2.

It is understood from this second auxiliary step 48 that the connection between the subcutaneous implant 2 and the communication relay 34 is governed by the computer server 38. In addition, the communication protocol issued by the computer server 38 allows the connections from the subcutaneous implant 2 to the communication relay 34 to be limited strictly when the subcutaneous implant 2 holds an information required by the computer server 38, or when the communication relay 34 holds an information configured for the subcutaneous implant 2.

The communication method 30 implements a second additional step 50, which takes place before the second auxiliary step 48, during which the communication relay 34 transmits an information relating to the detection of the subcutaneous implant 2 to the computer server 38. It is understood that this step in the communication method 30 is part of the process of controlling the connection of the subcutaneous implant 2 to the communication relay 34 by the computer server 38. The transmission of this detection signal by the communication relay 34 generates a request from the computer server 38 relating to the connection or the absence of connection between the communication relay 34 and the subcutaneous implant 2.

The authorization of the connection between the communication relay 34 and the subcutaneous implant 2 by the computer server 38 allows, in the embodiment shown, to implement a second step 52 of the communication method 30. During this second step 52 of the communication method 30, the subcutaneous implant 2 exchanges information with the computer server 38 by means of the communication relay 34. Alternatively, information may be exchanged between the computer server 38 and the subcutaneous implant 2 from the computer server 38 towards the subcutaneous implant 2. As mentioned above, such an exchange allows the subcutaneous implant 2 to be configured remotely.

The present invention achieves its stated aim by providing a system for monitoring at least one parameter representative of an episode of decompensation of a cardiac failure. This detection of said episode of cardiac decompensation is achieved by cross-referencing information obtained by a subcutaneous implant implanted in the patient. The subcutaneous implant communicates its data with a computer server by means of a communication relay which transitions the message to the computer server.

1. A system for monitoring at least one parameter representative of an episode of decompensation of a cardiac failure in a living being, the monitoring system comprising:
a subcutaneous implant configured to be introduced under the skin of said living being, the subcutaneous implant comprising at least one electrocardiograph and one accelerometer configured to collect data relating at least to the functioning of the heart of the living being, and
a computer server and a communication relay configured to allow at least the exchange of data collected by the subcutaneous implant with the computer server, the computer server being configured to calculate changes in at least one parameter, from among hemodynamic and/or respiratory and/or electrophysiological parameters, on the basis of the data collected by at least the accelerometer and the electrocardiograph,
the subcutaneous implant comprising communication means configured to communicate data collected by at least the accelerometer and the electrocardiograph by means of a low-energy Bluetooth connection.

2. The monitoring system as claimed in claim 1, wherein the communication means are configured to also communicate alerts relating to the availability of data collected by at least the accelerometer and the electrocardiogram communications separate from the data communications.

3. The monitoring system according to claim 1, wherein a communication protocol for the communication of data between the subcutaneous implant and the communication relay and/or the computer server is configured such that the communication of data from the subcutaneous implant towards the communication relay takes place in a plurality of successive data sending sessions.

4. The monitoring system according to claim 1, wherein a communication protocol for the communication of data between the subcutaneous implant and the communication relay is different from a communication protocol for the exchange of data between the communication relay and the computer server.

5. The monitoring system according to claim 1, wherein the subcutaneous implant comprises a temperature sensor configured to measure the body temperature of said living being.

6. The monitoring system according to claim 1, wherein the subcutaneous implant comprises at least one case on which at least a first electrode and a second electrode are arranged.

7. The monitoring system according to claim 1, wherein the computer server is configured to calculate at least one change relating to cardiac sounds and/or a pre-ejection period and/or a respiratory rate and/or a cardiac rate by means of the data collected by the subcutaneous implant.

8. The monitoring system according to claim 1, wherein the accelerometer is configured to calculate linear accelerations along three orthogonal axes, the computer server being configured to calculate changes in at least one hemodynamic and/or respiratory parameter on the basis in particular of a data of the accelerometer relating to at least one of said axes.

9. The monitoring system according to claim 1, wherein an algorithm is implemented on the computer server, said algorithm being configured to analyze the data acquired and transmitted by the subcutaneous implant so as to assess the risk of occurrence of an episode of decompensation of the cardiac failure.

10. A communication method for a monitoring system according to claim 1, the communication method comprising:
at least one first step during which the communication relay scans the surrounding space to detect the subcutaneous implant,
at least a first additional step during which the subcutaneous implant generates a warning signal specifying that data relating to the operation of the heart is capable of being exchanged with the computer server, and
at least one second step during which data or control instructions are exchanged between the subcutaneous implant and the computer server by means of the communication relay, the low-energy wireless communication network implemented for the communication between the subcutaneous implant and the communication relay being distinct from the high-speed wireless communication network implemented for the communication between the communication relay and the computer server.

11. The communication method as claimed in claim 10, wherein the communication method implements at least one second auxiliary step occurring before the second step and during which the communication relay detects the subcutaneous implant and sends to the computer server a request for identifying the subcutaneous implant, the computer server treating said identification request by sending to the subcutaneous implant, via the communication relay, an encryption key specific to said subcutaneous implant and a data retrieval request and/or a control instruction.

12. The communication method according to claim 10, wherein the communication between the communication relay and the subcutaneous implant is provided by a low-energy Bluetooth connection and the communication between the communication relay and the computer server is provided by a high-speed Internet connection.

13. The communication method according to claim 10, wherein the communication relay is configured to connect to a single subcutaneous implant, the communication relay being configured to continuously scan the surrounding space for seeking said subcutaneous implant.

14. The communication method according to claim 10, wherein the communication relay is configured to detect a plurality of subcutaneous implants, each subcutaneous implant being configured to allow or prevent the connection of the communication relay to said subcutaneous implant as a function of the recognition by said subcutaneous implant of an encryption key, said encryption key being unique to each subcutaneous implant, said encryption key being communicated by the computer server to said subcutaneous implants via the communication relay.
